(19) United States
(12) Patent Application Publication
(10) Pub. No.: US 2019/0167189 A1
(43) Pub. Date: Jun. 6, 2019

(54) SYSTEM FOR IMPROVING SAFETY

(71) Applicant: Alcohol Countermeasure Systems (International) Inc., Toronto (CA)

(72) Inventors: Paul Kuttner, Toronto (CA); Steven Leach, Toronto (CA); Matt Goledzinowski, Toronto (CA); Felix J.E. Comeau, Toronto (CA); Ali Shahidi Zandi, Toronto (CA); Azhar Quddus, Toronto (CA)

(21) Appl. No.: 16/212,133

(22) Filed: Dec. 6, 2018

Related U.S. Application Data
(60) Provisional application No. 62/595,359, filed on Dec. 6, 2017.
(60) Provisional application No. 62/696,524, filed on Jul. 11, 2018.

(51) Int. Cl. A61B 5/00; G06Q 99/00; A61B 5/18; A61B 5/145; A61B 5/08; A61B 5/16; A61B 3/11; G01N 33/487; G01N 33/94

(52) U.S. Cl. CPC A61B 5/4845; G06Q 99/00; A61B 5/18; A61B 5/145; G01N 33/948; A61B 5/082; A61B 5/163; A61B 3/112; G01N 33/487; A61B 5/14507

(57) ABSTRACT

A system for use in a jurisdiction comprises a legal regime applicable to the jurisdiction which provides, in a situation wherein persons can be obliged to submit to physiological/behavioural testing based upon need for capacity and at risk of sanction based upon the results of such physiological/behavioural testing, persons that have previously voluntarily undergone a capacity test and substantially simultaneously undergone physiological testing rights different from those who have not.

### CROSS-REFERENCE TO RELATED APPLICATION

This application claims the benefit of U.S. Provisional Patent Application Ser. No. 62/595,359 filed Dec. 7, 2017 and U.S. Provisional Patent Application Ser. No. 62/696,524 filed Jul. 11, 2018.

### BACKGROUND OF THE INVENTION

### 1. Field of the Invention

The invention relates to the field of impairment assessment.

### 2. Prior Art

Many jurisdictions are legalizing cannabis. This is a significant problem in the field of road safety since there exists no known way to confirm, on a beyond a reasonable doubt basis, inability of a human to operate a motor vehicle safely based upon a single technological test of that human. Many jurisdictions are adopting per se impairment standards based upon blood or saliva concentration, but regular users of marijuana can have relatively high blood/saliva concentrations with no evidence of impairment. As a result, judicial challenges are likely.

### SUMMARY OF THE INVENTION

Forming one aspect of the invention is a system for use in a jurisdiction. The system comprises a legal regime applicable to the jurisdiction which provides, in a situation wherein persons can be obliged to submit to physiological/behavioural testing based upon need for capacity and at risk of sanction based upon the results of such physiological/behavioural testing, persons that have previously voluntarily undergone a capacity test and substantially simultaneously undergone physiological/behavioural testing rights different from those who have not.

According to another aspect of the invention: the situation can be a roadside interaction between a police officer and a driver; the roadside physiological/behavioural test can result in information indicative of consumption of a drug by the person; and a roadside physiological/behavioural test of a person which results in information indicative of drug consumption by the person can be proof of an offence unless such person has previously undergone the substantially simultaneous testing.

According to another aspect of the invention: the situation can be a roadside interaction between a police officer and a driver; the roadside physiological/behavioural test can result in estimation of the concentration in the blood, breath or saliva of the person of a compound which is indicative of consumption of a drug; a roadside physiological/behavioural test of a person which results in a finding of a concentration of the compound in excess of a predetermined limit can be proof of an offence unless such person has previously undergone the substantially simultaneous testing and demonstrated capacity to drive under the same physiological/behavioural conditions.

According to another aspect of the invention: the situation can be a roadside interaction between a police officer and a driver; the roadside physiological/behavioural test can result in information indicative of consumption of a drug by the person and further information indicative of the capacity of the person; and a roadside physiological/behavioural test of a person which results in information indicative of consumption of drug consumption by a person can be proof of an offence unless such person has previously undergone the substantially simultaneous testing and demonstrated capacity to drive under the same physiological/behavioural conditions as obtained by the roadside test.

According to another aspect of the invention the further information can be measurements of eye movements, blinking and pupillometry.

According to another aspect of the invention, the drug can be alcohol or THC.

According to another aspect of the invention, capacity can be determined using the Tower of London test.

According to another aspect of the invention, the system can further comprise a plurality of centers at which a person can undergo a capacity test and substantially simultaneously undergo physiological/behavioural testing.

According to another aspect of the invention, the centers can issue credentials providing details of the results of the simultaneous testing.

### DETAILED DESCRIPTION OF THE PREFERRED EMBODIMENTS

The exemplary embodiment comprises a legal regime and a plurality of centers.

### Legal Regime

The legal regime provides, in a situation wherein a person is obliged to submit to physiological/behavioural testing based upon need for capacity and at risk of sanction based upon the results of such physiological/behavioral testing, persons that have previously voluntarily undergone a capacity test and substantially simultaneously undergone physiological/behavioural testing are provided rights different from those who have not.

More particularly: the situation is a roadside interaction between a police officer and a driver; the roadside physiological/behavioural test results in (i) information which is indicative of consumption of drug by the person; and (ii) further information indicative of the capacity of the person; and the legal regime is such that:
a roadside physiological/behavioural test which results in information indicative of drug consumption by a person is proof of an offence unless such person has previously undergone the substantially simultaneous testing;
a roadside physiological/behavioural test of a person which results in information indicative of drug consumption by a person is proof of an offence unless such person has previously undergone the substantially simultaneous testing and demonstrated capacity to drive under the same physiological/behavioural conditions as obtained by the test.

Even more particularly, the further physiological information is the concentration in saliva of a compound which is indicative of consumption of cannabis.

### Centers

At each of the plurality of centers, persons that wish to consume cannabis and minimize the likelihood of legal repercussions therefrom can attend and voluntarily undergo a capacity test and substantially simultaneously undergo physiological/behavioural testing in the presence of cannabis consumption.

Such person can, for example, at the center, at any time after consuming cannabis, provide a saliva sample and demonstrate capacity to drive a vehicle, based upon a Tower of London test.

Upon successfully demonstrating capacity, the center will provide a credential to the person indicating the concentration of the compound in the saliva of the compound indicative of cannabis consumption at which point capacity had been proven.

### Use

In use, in a situation such as a roadside spot check, the police may ask for the credential.

If the driver is unable to produce a credential, the officer may, based upon the laws of the jurisdiction, test the driver for the presence of any compound indicative of cannabis consumption, for example, through a saliva, blood, breath or urine test. If the compound is found present, the driver can be convicted of an offence.

If the driver produces a credential which demonstrates capacity of the driver at concentration levels at or above that at which the equipment of the police officer tests for [assuming the level of the compound increases with drug consumption], the officer may use that information to allow the driver to pass through the spot check without further investigation, particularly if the officer sees no obvious signs of criminal levels of impairment.

If the driver produces a credential which demonstrates capacity of the driver below concentration levels at which the equipment of the police officer tests for, the officer may call upon the driver to provide a saliva test. [For example, if the credential proves that the driver is capable at 10 ng THC/ml saliva, but the test equipment of the officer is adapted to provide confirmation if the concentration is at or above 15 ng THC/ml saliva, the saliva test may be called for.] If the driver tests below 15 ng THC/ml saliva, the officer may elect to refrain from further impairment testing of the driver, if the officer sees no obvious signs of criminal levels of impairment. If the driver tests above 15 ng THC/ml saliva, and the predetermined legal limit is at or above 15 ng THC/ml saliva, an offence will be deemed to have occurred.

### Advantage

Persons of ordinary skill will readily appreciate that the legalization of drug use as currently envisioned imposes great burdens on non-users in terms of public safety and policing costs. The system of the present invention ameliorates that problem by placing reasonable burdens upon persons that choose to consume drugs that are known to cause impairment. The system also facilitates the use of relatively low cost tests, such as Tower of London tests, eye tracking and “pass-fail” saliva testers, to be used for policing purposes.

### Variations

Whereas a specific embodiment is illustrated, persons of ordinary skill will appreciate that variations are possible.

For example, only, whereas THC is mentioned, the drug could be alcohol, cocaine, heroin, morphine, etc. Combinations of drugs could also be accommodated.

Further, whereas a Tower of London test is mentioned for the purpose of capacity determination at the center, other capacity determination tests could be utilized, such as driving simulators or the like.

Further, whereas the exemplary embodiment contemplates capture of concentration of a compound indicative of drug consumption using saliva, concentration could also be captured using blood or urine.

As well, the further physiological/behavioural information need not be tied to the presence of a drug or metabolite in the body but could be tied to physical tests that are themselves indicative of impairment such as the Tower of London test or eye tracking [pupillometry, blinking patterns, and gaze information such as HGN, VGN, and LOC]. In the case of physiological/behavioral testing, the centers would typically require a longer test, including an observed sleep period, a pre-consumption physical test to provide a baseline and post-consumption tests to permit correlation of capacity (for example, using a driving simulator) against the physical testing. In this specification, repeated mention is made of “physiological/behavioral”. It should be understood that this is intended to refer to either or both of physiological and behavioral, depending upon context. What is important is that a testing regime be selected which places reasonable obligations, upon persons that choose to consume drugs, to demonstrate capacity in situations where capacity is required.

Further, whereas roadside testing and the sanction of an offence for impaired driving is contemplated, the invention is not so limited. For example, the system could be used to test employees for fitness for work or to test drivers prior to enabling motion of a vehicle. For example, a test of the person could be carried out by the vehicle or the employer. If a drug is so detected, the system could call for a credential. Absent a credential, motion of the vehicle or access to the premises could be barred entirely; if a credential is presented, the driver or worker could, for example, be required to provide a satisfactory result on a Tower of London game prior to putting the vehicle in motion or commencing work and periodically thereafter, “satisfactory” being determined based upon previous testing of the driver/employee when not impaired by drug use.

Also, it will be appreciated that although it would often be ideal for the physiological/behavioral testing to be conclusive of impairment, this is not necessary, even in the context of criminal charges: in many cases, all that would need to be required to be established is a finding of “abnormal”. [Driving a vehicle is a privilege, as is consuming drugs that pose the potential for impairment: it is not at all unreasonable for society, if science does not permit the reliable determination of impairment but can reliably determine “normal”, to impose an obligation upon persons that consume impairment causing drugs to be functioning “normally”.]

Additionally, reference is made herein to an offence being established unless a person has previously undergone the substantially simultaneous testing and demonstrated capacity to drive under the same physiological/behavioural conditions as found at roadside; persons of ordinary skill will understand this “same” does not mean exactly the same. In the context of a physiological/behavioral measurement that increases with consumption of the drug, a person that has demonstrated capacity in the situation suitable therefor in the simultaneous testing at any measurement that is at or above the roadside level will be deemed capable; if the measurement decreases with consumption of the drug, a person that has demonstrated capacity in the situation suitable therefor in the simultaneous testing at any measurement that is below the roadside level will be deemed capable.

Further, whereas the specification references “calling for”, “production of” and “issuance of” credentials, it will be apparent that physical cards or the like are not required; a credential could be simply a record stored in a remote database and the credential could be produced, for example, by a police officer polling the database.

Accordingly, the invention should be understood to be limited only by the accompanying claims, purposively construed. What is claimed is:

1. A system for use in a jurisdiction, the system comprising:
a legal regime applicable to the jurisdiction which provides, in a situation wherein persons can be obliged to submit to physiological/behavioural testing based upon need for capacity and at risk of sanction based upon the results of such physiological/behavioural testing, persons that have previously voluntarily undergone a capacity test and substantially simultaneously undergone physiological testing rights different from those who have not.

2. A system according to claim 1, wherein:
the situation is a roadside interaction between a police officer and a driver;
the roadside physiological/behavioural test results in information indicative of consumption of a drug by the person; and
a roadside physiological/behavioural test of a person which results in information indicative of drug consumption by the person is proof of an offence unless such person has previously undergone the substantially simultaneous testing.

3. A system according to claim 1, wherein:
the situation is a roadside interaction between a police officer and a driver;
the roadside physiological/behavioural test results in the concentration in the blood, breath or saliva of the person of a compound which is indicative of consumption of a drug;
a roadside physiological/behavioural test of a person which results in a finding of a concentration of the compound in excess of a predetermined limit is proof of an offence unless such person has previously undergone the substantially simultaneous testing and demonstrated capacity to drive under the same physiological/behavioural conditions.

4. A system according to claim 1, wherein:
the situation is a roadside interaction between a police officer and a driver;
the roadside physiological/behavioural test results in information:
indicative of consumption of a drug by the person; and
further information indicative of the capacity of the person;
a roadside physiological/behavioural test of a person which results in information indicative of consumption of drug consumption by a person is proof of an offence unless such person has previously undergone the substantially simultaneous testing and demonstrated capacity to drive under the same physiological/behavioural conditions as obtained by the roadside test.

5. A system according to claim 4, wherein the further information indicative of capacity is measurements of eye movements, blinking and pupillometry.

6. A system according to claim 1, wherein the drug is alcohol or THC.

7. A system according to claim 1, wherein capacity is determined using the Tower of London test.

8. A system according to claim 1, further comprising a plurality of centres at which a person can undergo a capacity test and substantially simultaneously undergo physiological/behavioural testing.

9. A system according to claim 8, wherein the centres can issue credentials providing details of the results of the simultaneous testing.
